(19) United States
(12) Patent Application Publication
(10) Pub. No.: US 2022/0111381 A1
(43) Pub. Date: Apr. 14, 2022

(54) METHOD FOR MANUFACTURING 3D MICROFLUIDIC DEVICES

(71) Applicant: NETRI, Lyon (FR)

(72) Inventors: Florian LARRAMENDY, Lyon (FR); Thibault HONEGGER, Lyon (FR)

(21) Appl. No.: 17/426,991

(22) PCT Filed: Jan. 23, 2020
(86) PCT No.: PCT/FR2020/050097

§ 371 Date: Jul. 29, 2021

(30) Foreign Application Priority Data
Jan. 29, 2019 (FR) 19/00801

(51) Int. Cl. B01L 3/00; B33Y 80/00

(52) U.S. Cl. CPC B01L 3/502707; B01L 2200/12; B33Y 80/00

(57) ABSTRACT

Method for producing a microfluidic device, the method comprising a step of producing a master mould, the master mould comprising a first support member and a second support member, the second support member comprising a substrate and microstructures, the substrate having a first surface and a second surface opposite the first surface, the step of producing the master mould comprising the following sub-steps:—producing the second support member by forming the microstructures on the first surface of the substrate;—3D printing the first support member using a 3D printer, with a printing resin, the dimensions of the first support member being coordinated with the dimensions of the substrate in order to hold the substrate;—inserting the substrate of the second support member into the first support member.

### CROSS REFERENCE TO RELATED APPLICATIONS

This application is a National Stage of PCT Application No. PCT/FR2020/050097 filed on Jan. 23, 2020, which claims priority to French Patent Application No. 19/00801 filed on Jan. 29, 2019, the contents each of which are incorporated herein by reference thereto.

### TECHNICAL FIELD

The present invention concerns the field of microfluidic devices and particularly a method for manufacturing such devices.

### BACKGROUND

Microfluidic devices are used to replicate systems that manipulate small volumes of fluids, by using channels of the size of a few micrometers. For applications in biology, it is known to use the molding of polydimethylsiloxane (PDMS) on substrates on which micro-channels have been imprinted in photolithography to make two-dimensional devices. 3D printing could allow more complex three-dimensional devices to be made, but the resolution of 3D printing is not sufficient to produce the desired microfluidic devices, and the materials used in 3D printing are not compatible with the conditions of use of these devices for biological tests. On the other hand, the existing methods for manufacturing these devices are not very efficient in time, and cannot be adapted to a production in large quantities.

### BRIEF SUMMARY

The aim of the invention is therefore to provide a solution to all or part of these problems.

To this end, the present invention concerns a method for manufacturing a microfluidic device, the method comprising a step of producing a master mold, the master mold comprising a first support and a second support, the second support comprising a substrate and microstructures, the substrate having a first face and a second face opposite the first, the step of producing the master mold comprising the following sub-steps:
producing the second support by forming the microstructures on the first face of the substrate;
three-dimensional printing of the first support on a 3D printer, with a printing resin, the dimensions of the first support being adjusted to the dimensions of the substrate to contain the substrate;
inserting the substrate of the second support into the first support.

According to an embodiment, the invention comprises one or more of the following characteristics, alone or in combination.

According to an embodiment, the microstructures have at least one dimension less than 30 microns.

According to an embodiment, the substrate is made of silicon.

According to these arrangements, a master mold is produced by combining a 3D printing method for the production of patterns whose dimensions are submillimeter or millimeter and the micro-structuring, preferably by photolithography or micro-etching on a silicon substrate, or by any other equivalent method, for the formation of micrometric or submicrometric patterns.

According to an embodiment, the three-dimensional printing step of the first support comprises:
a sub-step of stopping the printing of the first support as a function of the height of the first printed support, before the step of inserting the substrate of the second support into the first support, and
a sub-step of continuing the printing of the first support from the height of the first support, the microstructures being aligned with printed patterns of the first support, the continuation sub-step being carried out after the insertion step.

According to an embodiment, the second support is partially encapsulated by the first support during the sub-step of continuing the 3-D printing of the first support.

According to these arrangements, the second support is better fixed to the first support, the second support no longer being able to move relative to the first support. On the other hand, the dimensions of the additional layer of the first support are not constrained by the dimensions of the second support which is encapsulated.

According to these arrangements, the silicon substrate is included. The silicon substrate is protected and is less likely to be broken when inserted into the first support. The different portions are aligned with each other by construction, and there is no interconnection problem between the different portions.

According to an alternative embodiment, the first support comprises a first portion and a second portion, the respective dimensions of the first portion and of the second portion being adjusted to the dimensions of the substrate of the second support so that the substrate of the second support is inserted into a recess of the first support, the recess being formed between the first portion and the second portion of the first support, the step of three-dimensional printing the first support comprises:
a sub-step of printing the first portion of the first support, and
a sub-step of printing the second portion of the first support.

This alternative embodiment has the advantage of overcoming some restrictions of exposure area of 3D printers.

According to the arrangements of this alternative embodiment, it is possible to create larger frames, within the limits of the dimensions imposed by the 3D printer. It is also possible to reuse the first support for several silicon substrates. No stopping step is necessary during 3D printing since everything is manually assembled.

According to an embodiment:
the first face and the second face of the substrate are spaced apart by a thickness of the substrate
the printing of the first support is stopped as soon as a total height, equal to a sum of a thickness of a bottom of the first printed support, added the thickness of the substrate of the second support, is greater than the height of the first printed support, by a value equal to or less than a predetermined threshold;
the inserting of the substrate of the second support into the first support rests the second face of the substrate on the bottom of the first support;
the method further comprising the following sub-step:
after the production of the second support, cutting the substrate from the second support around the microstructures, the dimensions of the first support being adjusted to the dimensions of the cut substrate, to contain the cut substrate.

According to an embodiment, the master mold production step further comprises the following sub-steps:
positioning of a 3D printer tool holder in a determined position, before the printing step;
extracting the tool holder from the 3D printer, after the stop printing step, and before the insertion step;
adding of resin on the first face of the substrate of the second support, after the insertion step;
positioning of the tool holder in the determined position, before continuing printing.

According to these arrangements, the step of inserting the second support into the first support is made easier, given the adjusted nature of the dimensions of the second support relative to those of the first support.

According to these arrangements, the step of adding resin on the surface of the silicon substrate prevents the appearance of bubbles during printing.

According to an embodiment, at the step of producing the second support, the formation of the microstructures comprises the implementation of one of the techniques from photolithography, wet or dry wafer etching, 2-photon technology, 3D printing with a resolution comparable to that of photolithography.

According to an embodiment, the dimensions of the first support are adjusted to the dimensions of the cut substrate, a width and a length of the first support being respectively greater than a width and a length of the substrate cut from the second support, by an equal value within a specified tolerance.

According to an embodiment, the tolerance margin for the width and the length of the first support is less than 0.25% of the width of the first support and less than 0.25% of the length of the first support, respectively.

According to an embodiment, the tolerance margin is determined as a function of the precision of the dimensioning of the microstructures formed on the second support, namely for example a determined tolerance margin of 100 μm.

According to an embodiment, the method further comprises a step of replicating the master mold to produce a first secondary mold from the master mold and to produce a second secondary mold from the first secondary mold.

According to an embodiment, the replication step comprises a sub-step of producing a first secondary mold and a sub-step of producing a second secondary mold, the sub-step of producing a first secondary mold comprising a sub-sub-step of positioning the master mold inside a container, and a sub-sub-step of depositing on the master mold, in the container, a first secondary material in the viscous phase.

According to an embodiment, the first secondary material is crosslinkable, the first secondary material preferably being a silicone rubber.

According to an embodiment, the sub-step of producing a first secondary mold further comprises a sub-sub-step of evacuating an internal volume of the container in which the master mold is positioned, and a sub-sub-step of annealing the first secondary material at room temperature for example, for example for 24 hours, before a sub-sub-step of removing the first secondary mold formed by the first deposited and crosslinked secondary material.

According to an embodiment, the first secondary material remains flexible in the crosslinked phase, in order to make it easier to remove, without destroying the master mold, the first secondary mold formed by crosslinking the first secondary material.

According to an embodiment, the first secondary material is a material compatible with the materials used to produce the master mold.

According to an embodiment, the sub-step of producing a second secondary mold comprises a sub-sub-step of depositing a second secondary material, for example in the liquid phase, on the first secondary mold, the second secondary material being crosslinkable and rigid in the solid crosslinked phase.

According to an embodiment, the sub-step of producing a second secondary mold comprises a sub-sub-step of removing the second secondary mold formed by the second crosslinked secondary material.

According to an embodiment, the second secondary material is a polyurethane resin.

According to an embodiment, the sub-step of producing a second secondary mold further comprises a sub-sub-step of removing bubbles in the second secondary material with a syringe cone, and a annealing sub-sub-step, for example at room temperature, for example for 2 hours, before the sub-sub-step of removing the second secondary mold.

According to an embodiment, the microfluidic device comprises at least one layer, and the method comprises:
a step of producing an encapsulation mold configured to cooperate with the second secondary mold in a production step of at least one layer, and
a step of producing at least one layer,

the step of producing an encapsulation mold comprising:
a 3D printing sub-step of a master encapsulation mold,
a sub-step of new replication of the master encapsulation mold to produce a secondary encapsulation mold and the encapsulation mold from the secondary mold encapsulation.

According to an embodiment, the new replication sub-step has the characteristics indicated above for the replication step, by starting from the master encapsulation mold to arrive at the encapsulation mold.

Thus according to an embodiment, the replication step comprises the production of a secondary encapsulation mold and of an encapsulation mold, the production of a secondary encapsulation mold comprising a positioning of the encapsulation master mold inside a container, and the deposition on the master encapsulation mold, in the container, of a first secondary material in viscous phase.

According to an embodiment, the first secondary material is crosslinkable, the first secondary material preferably being a silicone rubber.

According to an embodiment, the production of the secondary encapsulation mold further comprises an evacuation of an internal volume of the container in which the master encapsulation mold is positioned, and an annealing of the first secondary material, at ambient temperature for example, for example for 24 hours, before the removal of the secondary encapsulation mold formed by the first deposited and crosslinked secondary material.

According to an embodiment, the first secondary material remains flexible in the crosslinked phase, in order to make it easier to remove, without destroying the master encapsulation mold, from the secondary encapsulation mold formed by crosslinking the first secondary material.

According to an embodiment, the first secondary material is a material compatible with the materials used to produce the master encapsulation mold.

According to an embodiment, the production of an encapsulation mold comprises the deposition of a second secondary material, for example in liquid phase, on the secondary encapsulation mold, the second secondary material being crosslinkable and rigid in solid crosslinked phase.

According to an embodiment, the production of the encapsulation mold comprises the removing of the encapsulation mold formed by the second crosslinked secondary material.

According to an embodiment, the second secondary material is a polyurethane resin.

According to an embodiment, the production of the encapsulation mold further comprises the removal of bubbles in the second secondary material with a syringe cone, and an annealing, for example at room temperature, for example for 2 hours, before removal from the encapsulation mold.

According to an embodiment, the step of producing the at least one layer comprises:
a sub-step of molding the at least one layer by depositing a molding material, between the second secondary mold and the encapsulation mold, the encapsulation mold and the second secondary mold being pressed against each other so as to ensure surface contact between the top elements of the second secondary mold and a surface of the encapsulation mold,
a sub-step of annealing the material between the second secondary mold and the encapsulation mold, at a temperature and for a determined period of time.

According to an embodiment, the layer is thermoformed, on a first surface of the layer, in contact with the structures and/or microstructures present on the second secondary mold, and on a second surface of the layer, in contact with the structures and/or microstructures present on the encapsulation mold, the encapsulation mold itself having been previously obtained by the replication of a structured and/or microstructured encapsulation master mold.

According to an embodiment, the molding material is a PDMS.

According to an embodiment, the annealing temperature is about 80° C. and the annealing time is about 1 hour.

According to an embodiment, the at least one layer comprises at least two layers, the layers of the at least two layers being superimposed and fixed on each other after having been aligned with respect to one another so as to form a three-dimensional microfluidic device.

According to an embodiment, patterns of one of the at least two layers being aligned with patterns of another of the at least two layers to form nodes distributed in 3 dimensions and micro-channels, the micro-channels putting nodes in fluid com munication.

According to an embodiment, the layers are fixed on each other by being brought into contact with an oxygen plasma, for example for one minute.

According to an embodiment, the method comprises a first step of computer-aided design of the at least one layer according to a three-dimensional architecture of the microfluidic device, and a second step of computer-aided design of the master mold and of the master encapsulation mold according to the definition of the at least one layer.

According to one aspect of the invention, the invention also concerns a master mold for the manufacture of a microfluidic device, the master mold comprising:
a first support, produced by 3D printing, and
a second support, the second support comprising a substrate and microstructures, the substrate having a first face and a second face opposite to the first, the microstructures being formed on the first face of the substrate,

the dimensions of the first support being adjusted to the dimensions of the substrate to contain the substrate, the microstructures being aligned with printed patterns of the first support.

According to an embodiment, the master mold comprises one or more of the following characteristics, alone or in combination.

According to an embodiment, the microstructures have at least one dimension less than 30 microns.

According to an embodiment, the substrate is made of silicon.

According to an embodiment, the microstructures are formed by photolithography, or by a method having an equivalent resolution.

According to an embodiment, the second support is partially encapsulated by the first support.

According to these arrangements, the second support is better fixed to the first support, the second support no longer being able to move relative to the first support.

On the other hand, the dimensions of the additional layer of the first support are not constrained by the dimensions of the second support which is encapsulated.

According to an embodiment, the master mold is obtained by implementing the method according to the invention.

According to one aspect of the invention, the invention also concerns a flexible secondary mold for manufacturing a microfluidic device, the flexible secondary mold being a replication of the master mold according to one aspect of the invention in a first crosslinkable material which is flexible after having crosslinked.

According to an embodiment, the first material is compatible with the material of the master mold, and is deposited in the liquid or gel phase on the master mold, and is flexible after having crosslinked in the solid phase.

According to an embodiment, the first material is one of silicone rubber, polyurethane, polydimethylsolixane (PDMS), glue, elastomer, flexible foam, plastiline.

According to one aspect of the invention, the invention also concerns a rigid secondary mold for manufacturing a microfluidic device, the rigid secondary mold being a replication of the flexible secondary mold according to one aspect of the invention, in a second crosslinkable material, which is rigid after having crosslinked.

According to an embodiment, the second material is compatible with the material of the flexible secondary mold and is deposited in liquid or gel phase on the flexible secondary mold, and is rigid after having crosslinked in solid phase.

According to an embodiment, the second material is one of a polyurethane resin, a crosslinkable resin, a hardening gel, a hardening foam, a plastic, a glue.

According to one aspect of the invention, the invention also concerns a layer for the manufacture of a microfluidic device, the layer being a replication of the rigid secondary mold according to one aspect of the invention, in a third crosslinkable material.

According to an embodiment, the third material is compatible with the material of the rigid secondary mold, and is deposited in liquid or gel phase on the rigid secondary mold.

According to an embodiment, the third material is one of polyurethane, polydimethylsolixane (PDMS), silicone rubber, glue, elastomer, flexible foam, plastiline.

According to one aspect of the invention, the invention also concerns a microfluidic device comprising at least two layers according to one aspect of the invention, the at least two layers being placed and fixed on each other, patterns of the one of the at least two layers being aligned with patterns of another of the at least two layers to form nodes distributed in 3 dimensions and micro-channels, the micro-channels putting the nodes in fluid communication.

According to these arrangements, the master molds which do not require the formation of microstructures, that is to say structures whose smallest dimension is less than the resolution of the 3D printer, for example less than 30 pm, are produced by 3D printing, while those which require the formation of microstructures are produced by the method according to the invention, which combines 3D printing with photolithography or a method of precision and equivalent resolution.

According to these arrangements, the second secondary mold and the corresponding encapsulation mold, preferably rigid, can be rapidly reproduced, in large quantities and with the same level of detail as the master mold and the master encapsulation mold, respectively from the first secondary mold and secondary encapsulation mold, flexible of preferences.

Thus, the invention enables rapid prototyping because it uses the design and manufacturing advantages of 3D printing. The longest time is the manufacture of the master mold and the second support with its microstructures, about 24 hours, and the cavity in the form of the first flexible secondary mold, about 24 hours as well. The reproduction of the molds, in the form of the second, secondary, rigid molds takes only about 1 hour, and the molding of the layers of the microfluidic device takes only about 2 hours.

These last operations, less costly in time, can be carried out in parallel.

These arrangements make it possible to produce 3D microfluidic devices more efficiently, and therefore much more complex than conventional 2D devices.

The combination of two conventional technologies, 3D printing and photolithography or equivalent resolution method, makes it possible to obtain both the very high resolution, less than a micron, of the second method and the ease of use of 3D printing for the production of centimetric objects.

The initial equipment for manufacturing the devices is standard and therefore inexpensive, around 50 k€, compared to very high resolution 3D printing equipment, around 200 k€. In addition, the reproduction of molds and the manufacture of the devices are carried out with controlled quantities which make it possible not to have any loss of material, unlike the conventional methods for manufacturing such devices. The method according to the invention is therefore more economical.

The same structure of the different portions of the master molds, as well as the manufacturing method, can be standardized so that changes between each type of device have a minor impact on design and manufacturing time.

Compatibility between PDMS and 3D printing materials is not good, due to the problem of uncrosslinked PMDS. However, by going through intermediate steps with different materials, we can use the best material suitable for our application, in our case PMDS for biology, but we can consider other materials (silicones) for other applications.

Unlike the commercial 2D microfluidic devices currently sold, the devices produced cannot be reproduced by impression and remolding because a 3D impression would necessarily require the destruction of the molding during the extraction of the impression.

### BRIEF DESCRIPTION OF THE DRAWINGS

For its proper understanding, an embodiment of the invention is described with reference to the accompanying drawings representing, by way of non-limiting example, an embodiment of a device according to the invention. The same references in the drawings denote similar elements or elements having similar functions.

FIG. 1 is an illustration of the sub-steps of the production step of a master mold by 3D printing (F1a, F1b, F1c, F1d, F1e, F1f, F1g),

FIG. 2 is a representation of the respective dimensions of the first support and of the substrate of the second support (F2a, F2b),

FIG. 3 is a representation of a first portion and a second portion of the first support, according to a variant of the production step of a master mold by 3D printing,

FIG. 4 is an illustration of the sub-steps of a variant of the production step of a master mold by 3D printing (F4a, F4b, F4c, F4d, F4e, F4f),

FIG. 5 is a representation of the respective dimensions of the first support and the substrate of the second support according to the variant of the production step of a master mold by 3D printing,

FIG. 6 is an illustration of the sub-steps of the master mold replication step (F6a, F6b, F6c, F6d, F6e),

FIG. 7 is an illustration of the step of producing one layer of a microfluidic device (F7a, F7b), and the step of layering two layers together to form the 3D microfluidic device (F7c, F7d),

FIG. 8 is a schematic representation of the method according to the invention.

### DETAILED DESCRIPTION

The method according to the invention consists in coupling microstructured substrates with submillimeter or millimeter patterns directly printed and aligned by 3D printing in order to make a master mold, which can then be replicated to create «secondary» molds which will be used to mold the different layers of a final microfluidic device. The use of two successive molds allows a molding compatibility of the polydimethylsiloxane (PDMS) elastomer, which does not crosslink on the master mold, but only on a secondary mold.

By convention, a material is called crosslinkable if it is capable of crosslinking, i.e. if it is capable of passing from a pasty and viscous state of the material, to a solid state of the material, by polymerization of the material. By crosslinked phase, it is denoted the solid state of the material obtained after polymerization.

By convention, molding compatibility is defined here as the property of a material, for example PDMS, which allows the material to crosslink in contact with another material, that of a mold for example, without the contact between the material and the other material does not cause a chemical reaction or interference between the two materials. In this sense, it is important that the material of the secondary mold is compatible with PDMS.

The microfluidic device is split into several layers, each of which will be molded by said method. These PDMS layers will then be assembled by self-alignment to form a 3D microfluidic device.

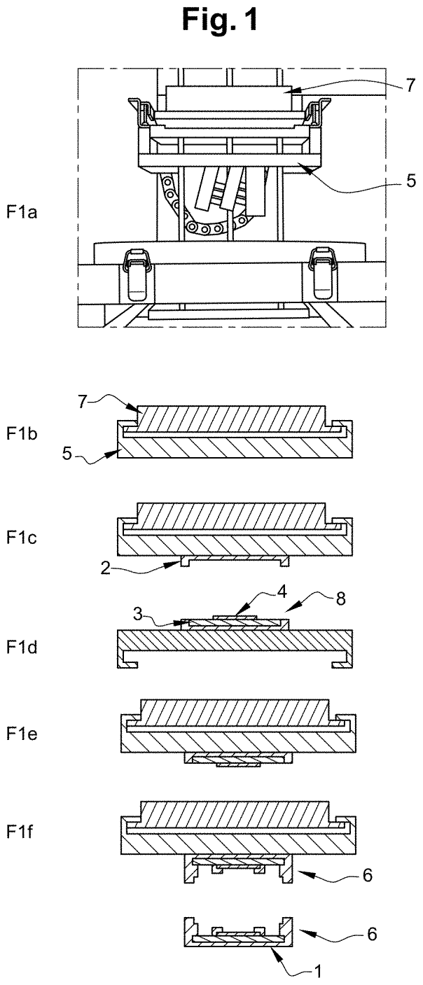

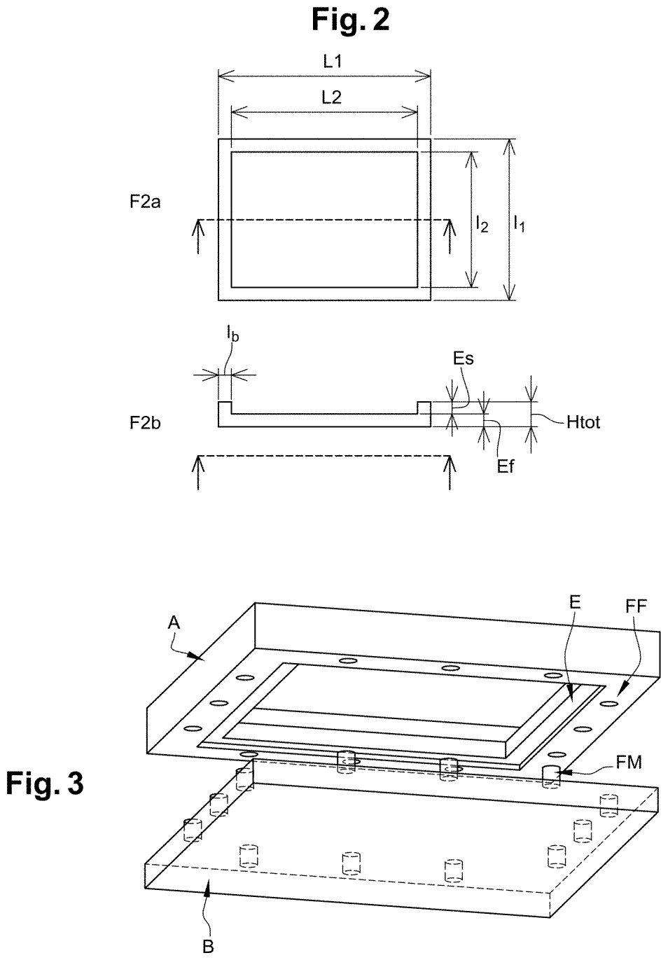

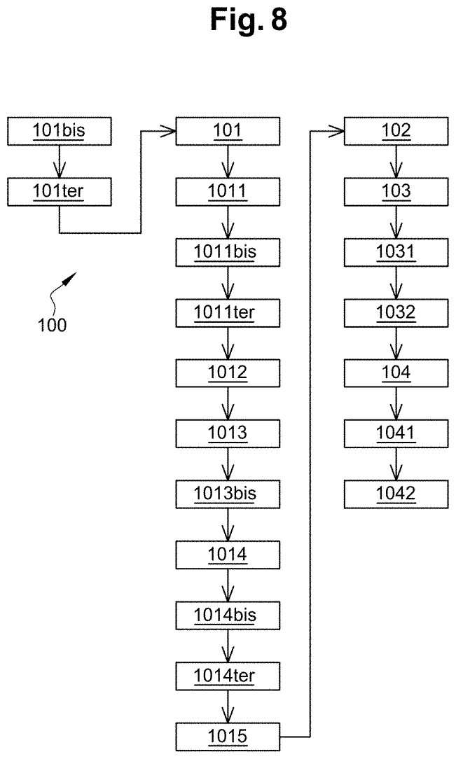

A detailed description of an embodiment of the method according to the invention will now be given, starting with the steps of producing a master mold, with reference to FIG. 1, which comprises various sub-figures F1a, F1b, F1c, F1d, F1e, F1f, and F1g and in FIG. 2, which also comprises different sub-figures F2a and F2b, as well as in FIG. 8.

The master mold 1, represented in F1g, comprises a first support 2 and a second support 8, the second support 8 comprising a substrate 3 and microstructures 4 formed on one face of the substrate 3.

The substrate 3 of the second support 8 is, for example, made of silicon.

During a first step 1011, the microstructures 4 are formed on the surface of one face of the substrate 3, for example using conventional techniques, for example silicon photolithography. The term «microstructures» means structured shapes which have at least one dimension less than 30 μm.

The production of the master mold then comprises the following steps:
printing 1012 in three dimensions of the first support 2 on a 3D printer 7, represented in FIG. 1 (F1a, and F1b to f); 3D printing is done for example with a printing resin, the dimensions of the first support 2 being adjusted to the dimensions of the substrate 3 of the second support 8, to allow the substrate 3 to be contained;
inserting 1014 of the substrate of the second support 8 in the first support.

According to an embodiment of the method according to the invention, the printing step 1012 in three dimensions (3D) of the first support 2, comprises:
a sub-step 1013 of stopping the 3D printing of the first support 2, as a function of a height of the first printed support 2, before the step 1014 of inserting the substrate of the second support 8 into the first support 2, and
a sub-step of continuing 1015 the printing of the first support from the height of the first support 2, the microstructures 4 being aligned with printed patterns of the first support 2, the 3D printing continuation sub-step being carried out after the insertion step 1014.

Thus, the 3D printing of the first support is stopped 1013, as illustrated in FIG. 1-F1c, to allow the insertion 1014 of the second support 8 in the first support 2. According to this embodiment, the stopping of the 3D printing is determined as a function of a height Hₜₒₜ of the edges of the first support. For example, when this height Hₜₒₜ is greater than the sum of a thickness E_{f} of the bottom of the first support 2 and a thickness of the substrate of the second support 8, then the 3D printing of the first support is interrupted, and the second support 8 is inserted inside the first support 2 before continuing the 3D printing of the first support 2.

Advantageously, the height Hₜₒₜ of the edges, when the 3D printing stops, will be sufficient to allow the substrate 3 to be encapsulated. Thus, it is possible to envisage stopping the 3D printing 1013 when the height Hₜₒₜ is equal to 3 mm, that is to say more than 99 3D printing levels for a 30 μm printing layer per level; this will allow sufficient stiffness of the edges to avoid the appearance of constraints on the substrate.

According to an embodiment, before the insertion 1014 of the second support 8 into the first support 2, the second support 8 is cut 1011 bis around microstructures 4, the dimensions of the first support 2 being adjusted to the dimensions of the cut substrate 3, to contain the cut substrate 3.

According to an embodiment, before the printing step 1012, a tool holder 5 of the 3D printer 7 is positioned 1011 ter at a determined position, reproducible after an extraction of the tool holder 5 from the 3D printer 7;

After the step of stopping printing 1013, the tool holder 5 is extracted 1013 bis from the 3D printer, as illustrated in FIG. 1-F1d, so as to facilitate the insertion 1014 of the second support 8 in the first support 2;

Resin can then be added 1014 bis on the first face of the substrate of the second support 8, after the insertion step 1014;

The tool holder 5 can then be repositioned to the position determined on the 3D printer 7, as illustrated in FIG. 1-F1c, before continuing the 3D printing 1015, as illustrated in FIG. 1-F1f,

According to an embodiment, the dimensions of the first support 2 are adjusted to the dimensions of the cut substrate 3; a width l₂ and a length L2 of the first support are respectively greater than a width and a length of the substrate cut from the second support 8, by a value equal to a determined tolerance margin. The tolerance margin for the width l₂ and the length L2 of the first support is typically less than 0.25% of the width l₂ of the first support and 0.25% of the length L2 of the first support, respectively.

According to an embodiment, the tolerance margin is determined as a function of the precision of the dimensioning of the microstructures formed on the second support 8. The tolerance margin may be for example 100 μm.

According to an alternative embodiment, or variant, which will now be described with reference to FIGS. 3, 4 and 5, the first support comprises a first portion A and a second portion B, the respective dimensions of the first portion A and of the second portion B being adjusted to the dimensions of the substrate of the second support so that the substrate of the second support fits into a recess E of the first support, the recess being formed between the first portion and the second portion of the first support.

The three-dimensional printing step of the first support comprises:
a 3D printing sub-step of the first portion A of the first support, and
a 3D printing sub-step of the second portion B of the first support.

This variant has the advantage of overcoming the restriction of the exposure area of 3D printers.

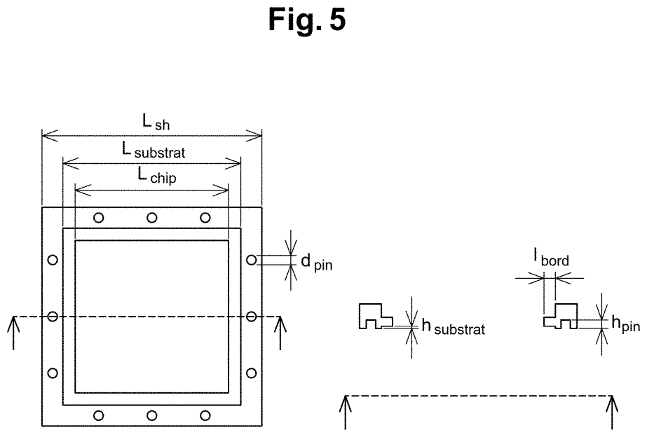

According to an embodiment of this variant, the dimensioning of the first portion A of the first support 2 is conditioned by the dimensions of the second support to be inserted. Thus:
the edges of the first portion A are of variable Ibord dimensions, advantageously identical, sufficiently wide for the second support to be strictly positioned in the center of the square including tolerance margins; these tolerances allow the adjusted insertion of the substrate of the second support to align the patterns to be printed in three dimensions, with the lithographed patterns; these tolerance margins also make it possible to take into account the constraints linked to the precision of the dimensions of the substrate. For example, in the configuration represented in FIG. 5, the lengths Lₛₕ of the substrate are selected to be identical to have a square device, and still by way of example, L_{chip}=35 mm, L_{substrat}=40 mm, Lₛₕ=50 mm and Lₛₕ=40 mm were selected. The insertion tolerance margin is therefore, in this case, constrained by the precision of the cutting of the silicon support, whose precision is 50 μm. This tolerance must be taken into account when designing the dimensioning of the photolithographed patterns, as illustrated in FIG. 5:
the embedding E of the substrate of the second support in the second portion B of the first support is done at a depth equivalent to the thickness of the substrate with a substrate tolerance h_{substrat} and an overlap l_{bord} of the second support. In the case presented here by way of example, the thickness of the substrate of the second support is h_{substrat}=550 μm and the overlap width of its edges is l_{bord}=2.5 mm.
female plugs FF are disposed on the periphery of the first portion A of the first support and are configured to receive male plugs FM disposed on the second portion B of the first support; each female plug FF has a diameter dₚᵢₙ=2 mm and a height hₚᵢₙ=2.1 mm taking into account a tolerance vis-à-vis the corresponding male plug FM.

The substrate of the second support is cut to the desired dimensions, for example with a disc saw.

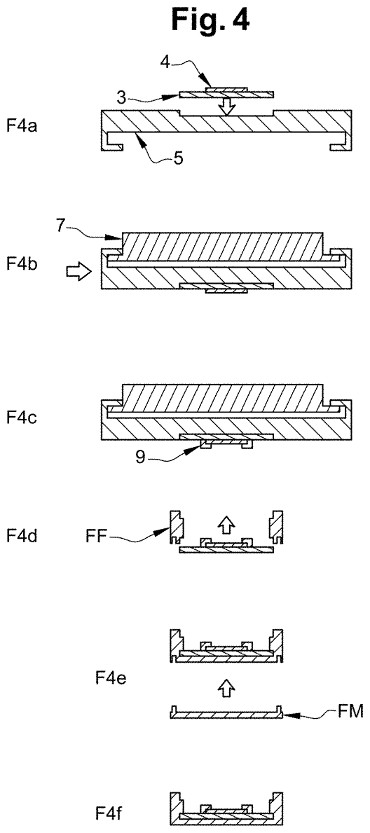

The positioning and fixing of the substrate of the second support in a recess dimensioned and made on the tool holder of the 3D printer, as illustrated in FIG. 4-F4a. The dimensions of the recess make it possible, for example, to receive the substrate from the second support with an accuracy of at least 50 μm in the plane of the tool holder, and with an accuracy of at least 5 μm in a plane perpendicular to the plane of the holder. If the printer has a surface detection mode, there is no need to have a suitable tool holder as the surface of the silicon holder will be used as a reference.

The tool holder is positioned in a configuration that allows the tool holder to be aligned with the pattern to be printed. It can for example be pushed to the stop, as shown in FIG. 4-F4b.

The 3D printing of the patterns 9 of the first support are printed with the 3D printer, as illustrated in FIG. 4-F4c.

After having detached the tool holder from the 3D printer, and after having detached the second support from the tool holder, with the 3D printed patterns 9, the second support, with the 3D printed patterns 9, is positioned in the anchor of the first portion A of the first support, between the first portion A and the second portion B of the first support, as illustrated in FIGS. 4-F4d, 4-F4e and 4-F4f.

The male plugs FM of the second portion B of the first holder are configured to penetrate the female plugs FF of the first portion A of the first holder, to encapsulate the substrate of the second holder, as illustrated in FIGS. 4-F4d, 4-F4e and 4-F4f.

Thus, according to the embodiment which has just been described, the substrate of the second support, with the 3D printed patterns, is anchored in the first portion A of the first support, while the second portion B of the first support serves as a wrapper. However, according to another variant, the second portion B, instead of the first portion A, of the first support, can serve as a wrapper, while the first portion A of the first support is configured to embed the substrate of the second support therein.

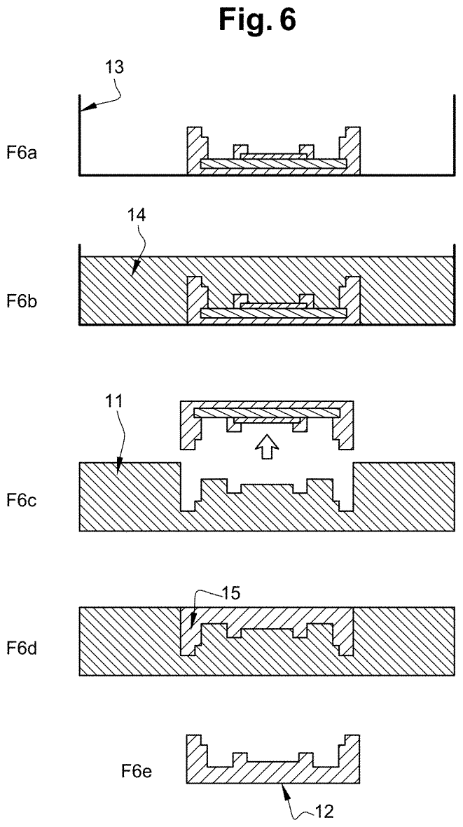

After the steps of producing a master mold, which have just been described, according to several embodiments and with reference to FIGS. 1, 2, 3, 4, 5 and 8, the method according to the invention further comprises, according to a complementary embodiment, a step 102 of replicating the master mold 1 to produce a first secondary mold 11 from the master mold 1 and to produce a second secondary mold 12 from the first secondary mold 11. This step 102 of replicating the master mold will now be described in detail, with reference to Figures F6a, F6b, F6c, F6d, F6e in FIG. 6.

According to an embodiment, the sub-step of producing a first secondary mold comprises a sub-sub-step, illustrated in FIG. 6-F6a, of positioning the master mold inside a container 13, and a sub-sub-step, illustrated in FIG. 6-F6b, of depositing on the master mold, in the container 13, a first secondary material 14 in the viscous phase.

The first secondary material 14 is a crosslinkable material, preferably a silicone rubber.

The sub-step of producing a first secondary mold 11 further comprises a sub-sub-step of evacuating an internal volume of the container 13 in which the master mold is positioned, and a sub-sub-step of annealing the first secondary material 14, at ambient temperature for example, for example for 24 hours, before a sub-sub-step, illustrated in FIG. 6-F6c, of removing the first secondary mold 11 formed by the first deposited and crosslinked secondary material 14.

According to an embodiment, the first secondary material 14 remains flexible in the crosslinked phase; this makes it easier to remove, without destroying the master mold, the first secondary mold 11 formed by crosslinking the first secondary material 14.

According to an embodiment, the first secondary material 14 is a material compatible with the material used to produce the master mold.

According to an embodiment, the sub-step of producing a second secondary mold 12 comprises a sub-sub-step, illustrated in FIG. 6-F6d, of depositing a second secondary material 15, for example in liquid phase, on the first secondary mold 11, the second secondary material 15 being crosslinkable, and rigid in the solid crosslinked phase.

According to an embodiment, the sub-step of producing a second secondary mold 12 comprises a sub-sub-step, illustrated in FIG. 6-F6e, of removing the second secondary mold 12 formed by the second crosslinked secondary material 15.

According to an embodiment, the second secondary material 15 is a polyurethane resin.

According to an embodiment, the sub-step of producing a second secondary mold 12 further comprises a sub-sub-step of removing bubbles in the second secondary material 15 with a syringe cone, and an annealing sub-sub-step, for example at room temperature, for example for 2 hours, before the sub-sub-step of removing the second secondary mold 12.

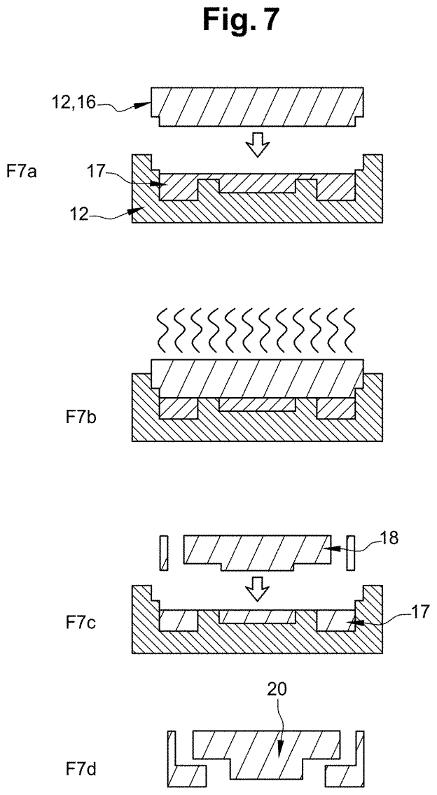

After the step 102 of replicating the master mold 1 to produce a first 11 and then a second 12 secondary mold, from the master mold 1, the method according to the invention further comprises, according to a complementary embodiment illustrated by FIG. 7, a step 104 of producing one or more layers 17, 18; the layers 17, 18 are intended to be superimposed to form a microfluidic circuit 20.

According to an embodiment, the step 104 of producing one or more layers 17, 18 is preceded by a step 103 of producing an encapsulation mold 16, illustrated in FIG. 7-F7a.

The production step 103 of an encapsulation mold 16 comprises:
a sub-step 1031 of 3D printing of a master encapsulation mold,
a sub-step 1032 of new replication of the master encapsulation mold to produce a secondary encapsulation mold and the encapsulation mold 16 from the secondary encapsulation mold;

According to an embodiment, the new replication sub-step 1032 has the characteristics indicated above for the replication step, starting from the master encapsulation mold to arrive at the encapsulation mold 12, 16

Thus, according to an embodiment already described and illustrated in FIG. 6, the new replication step comprises the production of a secondary encapsulation mold 11 and of an encapsulation mold 12, 16, the production of a secondary encapsulation mold 11 comprising a positioning of the master encapsulation mold 1 inside a container 13, and the deposition on the master encapsulation mold 1, in the container 13, of a first secondary material 14 in viscous phase.

According to an embodiment, the first secondary material 14 is crosslinkable, the first secondary material 14 preferably being a silicone rubber.

According to an embodiment, the production of the secondary encapsulation mold 11 further comprises an evacuation of an internal volume of the container 13 in which the master encapsulation mold 1 is positioned, and an annealing of the first secondary material 14, at room temperature for example, for example for 24 hours, before the removal of the secondary encapsulation mold 11 formed by the first deposited and crosslinked secondary material 14.

According to an embodiment, the first secondary material 14 remains flexible in the crosslinked phase, in order to make it easier to remove, without destroying the master encapsulation mold 1, from the secondary encapsulation mold 11 formed by crosslinking the first secondary material 14.

According to an embodiment, the first secondary material 14 is a material compatible with the materials used to produce the master encapsulation mold 1.

According to an embodiment, the production of an encapsulation mold 12, 16 comprises the deposition of a second secondary material 15, for example in liquid phase, on the secondary encapsulation mold 11, the second secondary material 15 being crosslinkable and rigid in the solid crosslinked phase.

According to an embodiment, the production of the encapsulation mold 12, 16 comprises the removal of the encapsulation mold 12, 16 formed by the second crosslinked secondary material 15.

According to an embodiment, the second secondary material 15 is a polyurethane resin.

According to an embodiment, the production of the encapsulation mold 12, 16 further comprises the removal of bubbles in the second secondary material 15 with a syringe cone, and an annealing, for example at room temperature, for example for 2 hours, before removing the encapsulation mold 12, 16.

The production step 103 of the encapsulation mold 12, 16 is followed by a production step 104 of a first layer 17 of the microfluidic device 20. As illustrated in FIG. 7, the production step 104 comprises:
a sub-step 1041 of molding at least one layer, produced by depositing a molding material, between the second secondary mold 12 and the encapsulation mold 12,16, the encapsulation mold 12,16 and the second secondary mold 12, being pressed against each other so as to ensure surface contact between the upper elements of the second secondary mold 12 and a lower surface of the encapsulation mold 12, 16;
an annealing sub-step 1042 of the molding material between the second secondary mold 12 and the encapsulation mold 12, 16, at a determined temperature and for a period of time.

According to an embodiment, the molding material is a PDMS.

According to an embodiment, the annealing temperature is about 0° C. and the annealing time is about 1 hour.

The previously described steps of the method according to the invention are repeated as many times as there are layers 17, 18 to be produced to form the microfluidic circuit 20.

The layers 17, 18 are superimposed and fixed to each other after being aligned relative to each other to form the three-dimensional microfluidic device.

According to an embodiment, patterns of one of the at least two layers being aligned with patterns of another of the at least two layers to form nodes distributed in 3 dimensions and micro-channels, the micro-channels putting nodes in fluid communication.

According to an embodiment, the layers are fixed on each other by being brought into contact with an oxygen plasma, for example for one minute.

According to an embodiment, the layers 17, 18 of the microfluidic device 20 are defined during a first step 101 bis of computer-aided design, as a function of a three-dimensional architecture of the microfluidic device 20; the master mold 1 and the master encapsulation mold are defined for each layer 17, 18 in a second step 101 ter of computer-aided design.

According to one aspect, the invention concerns a master mold 1, obtained by the method according to the invention and comprising:
a first support 2, produced by 3D printing, and
a second support 8, the second support comprising a substrate 3 and microstructures 4, the substrate 3 having a first face and a second face opposite the first, the microstructures being formed on the first face of the substrate 3, the dimensions of the first support 2 being adjusted to the dimensions of the substrate 3 to contain the substrate 3, the microstructures 4 being aligned with printed patterns of the first support 2.

According to an embodiment, the microstructures have at least one dimension less than 30 microns.

According to an embodiment, the substrate is made of silicon.

According to an embodiment, the microstructures are formed by photolithography, or by a method having an equivalent resolution.

The master mold is obtained by implementing the method according to the invention.

According to another aspect, the invention also concerns a flexible secondary mold 11 for the manufacture of a microfluidic device 20, the flexible secondary mold 11 being a replication of the master mold 1 in a first crosslinkable material which is flexible after having crosslinked.

According to an embodiment, the first crosslinkable material which is flexible after having crosslinked is one of silicone rubber, polyurethane, elastomer, flexible foam, plastiline.

According to another aspect, the invention also concerns a rigid secondary mold 12 for the manufacture of a microfluidic device 20, the rigid secondary mold 12 being a replication of the flexible secondary mold 11 according to one aspect of the invention, in a second crosslinkable material, which is rigid after having crosslinked.

According to an embodiment, the second crosslinkable material which is rigid after having crosslinked is one of a polyurethane resin, a crosslinkable resin, a hardening gel, a hardening foam, a plastic, a glue.

According to another aspect, the invention also concerns a layer 17, 18 for the manufacture of a microfluidic device 20, said layer 17, 18 being a replication of the rigid secondary mold 11, in a third crosslinkable material.

According to an embodiment, the third crosslinkable material is one of a polydimethylsolixane (PDMS), a silicone, an adhesive, an elastomer, a flexible foam, a plastiline.

According to another aspect, the invention also concerns a microfluidic device 20 comprising at least two layers 17, 18 according to one aspect of the invention, the at least two layers 17, 18 being placed and fixed on each other, patterns of one of the at least two layers being aligned with patterns of another of the at least two layers to form nodes distributed in 3 dimensions and micro-channels, the micro-channels putting the nodes in fluid communication.

According to these arrangements, the master molds, which do not require the formation of microstructures, that is to say structures whose smallest dimension is less than the resolution of the 3D printer, for example less than 30 μm, are produced by 3D printing, while those which require the formation of microstructures are produced by the method according to the invention, which combines 3D printing with photolithography or a method of precision and equivalent resolution.

According to these arrangements, the second secondary mold and the corresponding encapsulation mold, preferably rigid, can be rapidly reproduced, in large quantities and with the same level of detail as the master mold and the master encapsulation mold, respectively from the first secondary mold and secondary encapsulation mold, preferably flexible.

Thus, the invention enables rapid prototyping, because it uses the design and manufacturing advantages of 3D printing. The longest time is the manufacture of the master mold and the second support with its microstructures, about 24 hours, and the printing in the form of the first flexible secondary mold, about 24 hours as well. The reproduction of the molds, in the form of the second, rigid secondary molds takes only about 1 hour, and the molding of the layers of the microfluidic device takes only about 2 hours.

These last operations, less costly in time, can be carried out in parallel.

1. A method for manufacturing a microfluidic device, the method comprising a step of producing a master mold, the master mold comprising a first support and a second support, the second support comprising a substrate and microstructures, the substrate having a first face and a second face opposite to the first, the step of producing the master mold comprising the following sub-steps:
producing of the second support by forming the microstructures on the first face of the substrate;
three-dimensional printing of the first support on a 3D printer, with a printing resin, the dimensions of the first support being adjusted to the dimensions of the substrate to contain the substrate;
inserting the substrate of the second support in the first support.

2. The method according to claim 1 wherein the step of three-dimensional printing the first support on a 3D printer, comprises:
a sub-step of stopping the printing of the first support as a function of a height of the first printed support, before the step of inserting the substrate of the second support in the first support, and
a sub-step of continuing the printing of the first support from the height of the first support, the microstructures being aligned with printed patterns of the first support, the sub-step of continuing the printing of the first support from the height of the first support being carried out after the step of inserting the substrate of the second support in the first support.

3. The method according to claim 2, wherein:
the first face and the second face of the substrate are separated by a thickness of the substrate,
the printing of the first support is stopped as soon as a total height, equal to a sum of a thickness of a bottom of the first printed support, added to the thickness of the substrate of the second support, is greater than the height of the first printed support, by a value equal to or less than a predetermined threshold;
the inserting of the substrate of the second support in the first support rests the second face of the substrate on the bottom of the first support;
the method further comprising the following sub-step:
after production of the second support, cutting the substrate of the second support around microstructures, the dimensions of the first support being adjusted to the dimensions of the cut substrate, to contain the cut substrate.

4. The method according to claim 2, wherein the producing step of the master mold further comprises the following sub-steps:
positioning of a tool holder of the 3D printer at a determined position, before the three-dimensional printing step;
extracting the tool holder of the 3D printer, after the stop printing step, and before the inserting step;
adding resin on the first face of the substrate of the second support, after the inserting step; and
positioning the tool holder in the determined position, before continuing printing.

5. The method according to claim 1, wherein, in step of producing the second support, the formation of the microstructures comprises the implementation of one of the techniques among photolithography, wet or dry wafer engraving, 2-photon technology, 3D printing with a resolution comparable to that of photolithography.

6. The method according to claim 1, further comprising a step of replicating the master mold to produce a first secondary mold from the master mold and to produce a second secondary mold from the first secondary mold.

7. The method according to claim 6, wherein the microfluidic device comprises at least one layer, the method comprising:
a step of producing an encapsulation mold configured to cooperate with the second secondary mold in a production step of the at least one layer (17, 18), and
a production step of at least one layer, the production step of an encapsulation mold comprising:
a sub-step of 3D printing an encapsulation master mold,
a sub-step of further replicating the master encapsulation mold to produce a secondary encapsulation mold and the encapsulation mold from the secondary encapsulation mold.

8. The method according to claim 7, wherein the step of producing the at least one layer comprises:
a sub-step of molding at least one layer by depositing a molding material, between the second secondary mold and the encapsulation mold, the encapsulation mold and the second secondary mold being pressed against each other so as to ensure a surface contact between upper elements of the second secondary mold and a surface of the encapsulation mold,
an annealing sub-step of the molding material between the second secondary mold and the encapsulation mold, at a temperature and for a determined period.

9. The method according to claim 7, wherein the at least one layer comprises at least two layers, each layers of the at least two layers being superimposed and fixed on each other after being aligned with respect to each other so as to form a three-dimensional microfluidic device.

10. The method according to claim 7, wherein the method comprises a first step of computer-aided design of the at least one layer according to a three-dimensional design of the microfluidic device, and a second step of computer-aided design of the master mold and of the encapsulation master mold according to a definition of the at least one layer.

11. A master mold for the manufacture of a microfluidic device, the master mold comprising:
a first support, produced by 3D printing,
and a second support, the second support comprising a substrate and microstructures, the substrate having a first face and a second face opposite to the first, the microstructures being formed on the first face of the substrate,
the dimensions of the first support being adjusted to the dimensions of the substrate to contain the substrate, the microstructures being aligned with printed patterns of the first support.

12. A flexible secondary mold for the manufacture of a microfluidic device, the flexible secondary mold being a replication of the master mold according to claim 11 in a first crosslinkable material which is flexible after having crosslinked.

13. A rigid secondary mold for the manufacture of a microfluidic device, the rigid secondary mold being a replication of the flexible secondary mold according to claim 12, in a second crosslinkable material, which is rigid after having crosslinked.

14. A layer for the manufacture of a microfluidic device, the layer being a replication of the rigid secondary mold according to claim 13, in a third crosslinkable material.

15. A microfluidic device comprising at least two layers according to claim 14, the at least two layers being placed and fixed on each other, patterns of one of at least two layers being aligned with patterns of another of the at least two layers to form nodes distributed in 3 dimensions and micro-channels, the micro-channels putting the nodes in fluid communication.

16. The method according to claim 3, wherein the producing step of the master mold further comprises the following sub-steps:
positioning of a tool holder of the 3D printer at a determined position, before the three dimensional printing step;
extracting the tool holder of the 3D printer, after the stop printing step, and before the inserting step;
adding resin on the first face of the substrate of the second support, after the inserting step;
positioning the tool holder in the determined position, before continuing printing.

17. The method according to claim 16, wherein, in step of producing the second support, the formation of the microstructures comprises the implementation of one of the techniques among photolithography, wet or dry wafer engraving, 2-photon technology, 3D printing with a resolution comparable to that of photolithography.

18. The method according to claim 2, wherein, in step of producing the second support, the formation of the microstructures comprises the implementation of one of the techniques among photolithography, wet or dry wafer engraving, 2-photon technology, 3D printing with a resolution comparable to that of photolithography.

19. The method according to claim 3, wherein, in step of producing the second support, the formation of the microstructures comprises the implementation of one of the techniques among photolithography, wet or dry wafer engraving, 2-photon technology, 3D printing with a resolution comparable to that of photolithography.

20. The method according to claim 4, wherein, in step of producing the second support, the formation of the microstructures comprises the implementation of one of the techniques among photolithography, wet or dry wafer engraving, 2-photon technology, 3D printing with a resolution comparable to that of photolithography.
